(19) United States
(12) Patent Application Publication
(10) Pub. No.: US 2023/0103225 A1
(43) Pub. Date: Mar. 30, 2023

(54) Method and System for Delivering Intervention Based on User Status

(71) Applicant: Docbotic, Inc., Wilmington, DE (US)

(72) Inventors: Jenna Xu, Chicago, IL (US); Chelsey Wilks, St.Louis, MO (US)

(21) Appl. No.: 17/941,241

(22) Filed: Sep. 9, 2022

Related U.S. Application Data
(60) Provisional application No. 63/242,646, filed on Sep. 10, 2021.

(51) Int. Cl. G16H 20/70; G16H 10/60

(52) U.S. Cl. CPC G16H 20/70; G16H 10/60

(57) ABSTRACT

The claimed invention relates to implementing psychotherapy intervention as an application on a remote telecommunications device that provides behavioral feedback to the user in order to prevent conditions that cause negative outcomes.

### PRIORITY CLAIM

This utility application claims priority to and incorporates by reference U.S. Prov. App. No.. 63242646 filed on Sep. 10, 2021.

### FIELD OF INVENTION AND BACKGROUND

The claimed invention relates to implementing remote psychotherapy intervention using telecommunications systems that provide well-timed behavioral feedback to prevent conditions that cause negative outcomes.

### SUMMARY

Many types of psychotherapy focus on assessment to try and better capture the progression of mental illnesses. Typically, this is done through therapy sessions where the therapist interviews the patient. However, this is time consuming and expensive. The present invention does not employ therapists or teletherapy . Instead, it is a technology that acts as a well-timed intervention to the patient’s behavior based on codified, evidence-based therapeutic protocols. The computer system implementing the method performs steps that are selected based on behavioral data and user interaction in order to track the status of the user while at the same time delivering feedback and recommendations at relevant times throughout the day to the user . Another aspect of the claimed invention is to detect factors that may relate to an unfavorable outcome or make predictions in addition to ameliorative action. The claimed invention in one embodiment uses a software application operating on a telecommunications device that the patient interacts with which automatically provides feedback to the patient, in order to encourage the patient to make changes in behavior as well as to identify factors in the patient interactivity data that predict potentially negative outcomes. In another embodiment, the interaction between the user and the system is by the system transmitting SMS or similar texting messages and the user responding to those messages.

### DESCRIPTION OF THE FIGURES

The headings provided herein are for convenience only and do not necessarily affect the scope or meaning of the claimed invention. In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced (e.g., element 101 is first introduced and discussed with respect to FIG. 1).

FIG. 1: Exemplary flowchart depicting an interactive script graph for tracking of the patient’s sleep patterns

FIG. 2: Exemplary flowchart depicting an interactive script graph for tracking of the patient’s alcohol use and nap patterns for insomnia tracking

FIG. 3: Exemplary flowchart depicting an interactive script for tracking the patient’s intervention process

FIG. 4: Exemplary system architectural overview of the claimed invention

FIG. 5: Flowchart for commencing patient status check on a daily basis.

FIG. 6: Flowchart for determining next prompt in the interactive script

FIG. 7: Flowchart for processing next prompt and tracking it.

FIG. 8: Flowchart for transmitting prompt to patient phone.

FIG. 9: Flowchart for receiving response to transmitted prompt from patient phone.

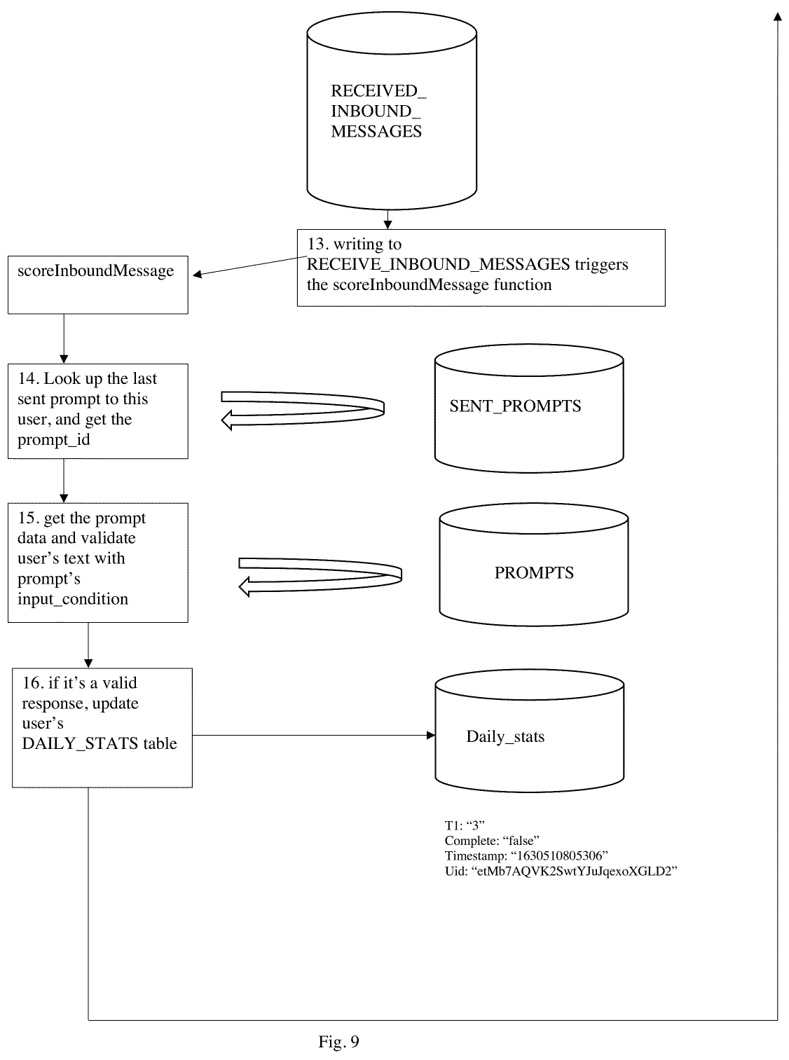

FIG. 10: Example data structure for storing patient status and response

FIG. 11: Example data structure for encoding a node in the interactive script graph.

FIG. 12: Example data structure for encoding a node in the interactive script graph

### DETAILED DESCRIPTION

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other features not described in detail herein. Additionally, some well-known structures or functions may not be shown or described in detail below, so as to avoid unnecessarily obscuring the relevant description. The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

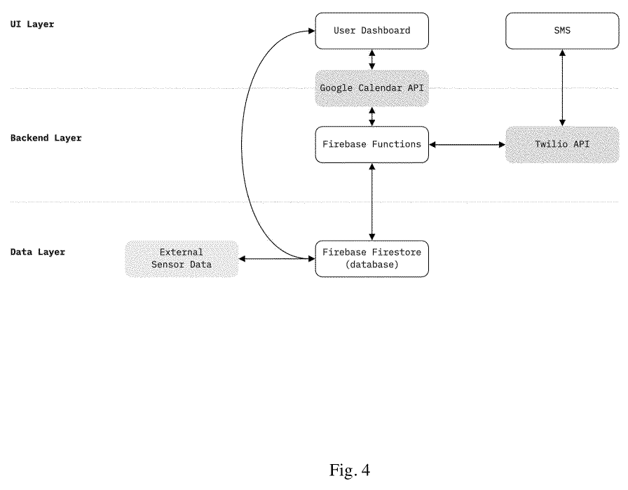

In a preferred embodiment, the user signs up and logs into an application running on their telecommunication device, for example, a smart-phone, connected to a server via a telecommunications network. The system embodies the claimed invention. FIG. 4. When launched the system at the server receives an identifier data from the app operating on the user’s mobile device or computer or by means of receiving an SMS message from a known phone number. That identifier is validated in order to maintain security over the interactive data already stored in the system and any new data that is input by the user. In addition, the security of the app is checked to ensure that no one but the actual patient is able to modify the interactivity data in the system that is associated with the patient.

Once validated, the system queries its database using the patient identifier in order to determine the current state of the patient. The possible states of the patient is represented by a set of data values stored in the computer system. The range of possible states may be represented by a graph, and each node in the graph relates to its neighbors in that the logical inputs into the system determine if one state is moved to another state thereby re-assigning the data values representing the state value. The patient state may be one or more data values, or a vector, indicating characteristics about the patient’s interaction with the app. In one embodiment, the state vector of a patient will have a first element that encodes which of several possible interactivity script graphs the patient is traversing. The vector may also have a second element that encodes the location of a node in that interactivity script graph where the patient is located. An example patient script graph is presented below:

PatientStateVector<PatientID, InteractivityScriptGraph, CurrentNode>

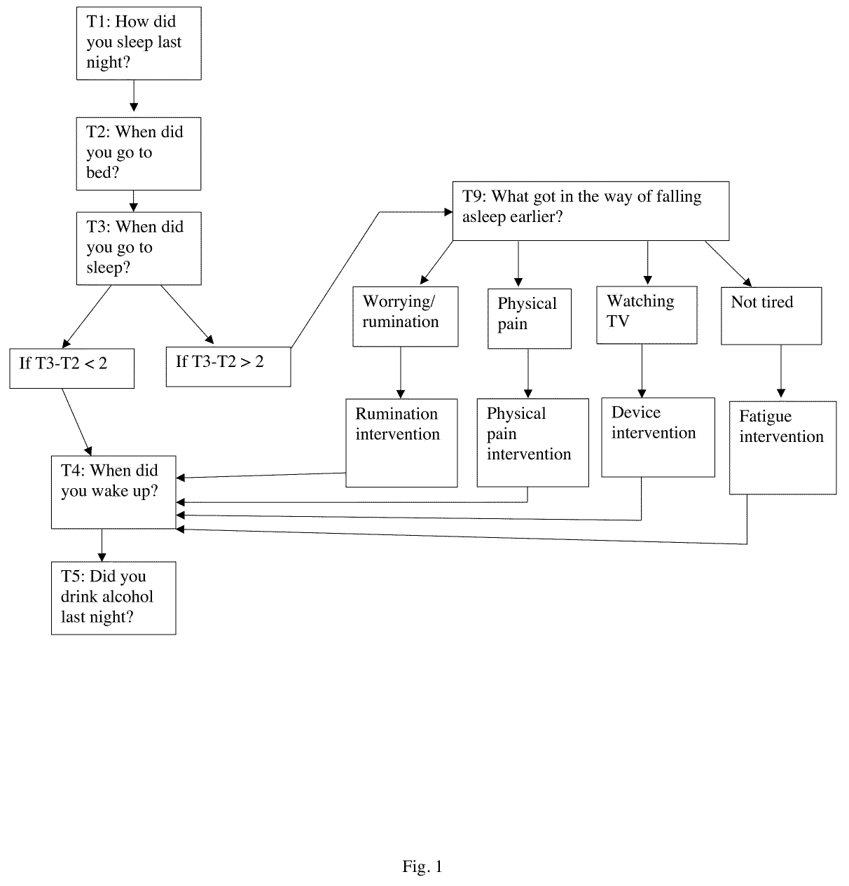

In yet another embodiment, each session starts at a start point in the interactivity script graph. The system in this way determines a start node when the session begins. An example script graph is shown in FIG. 1.

The system then transmits to the mobile device running on the user’s mobile device a text string corresponding to the current node location. The mobile device then receives from the user a response, either as a button press (e.g. yes or no) or numbers, text, or other data input, including haptic feedback. The mobile device then transmits these responses to the server. The server stores the responses in an interaction data file corresponding to the patient in order that all interaction is stored in a record. In one embodiment, the transmitted text is stored in the patient’s data record with a time stamp, and the received response is stored with its own time stamp. This file may be formatted as an XML file in order that each interaction with the patient and its time stamp is stored in a structured way. In one embodiment, the interaction may be encoded as follows:

Question<InteractivityGraph, NodeIndex, TextString, TimeStamp> Response<PatientID, InteractivityGraph, NodeIndex, RecievedData, TimesStamp>

The server operates a process that is a graph traversing engine that then takes as input the current node location and the data input received from the mobile device in order to determine a next node in the interactivity graph. The engine will use Boolean logic to apply a logic rule to an input variable string comprised of the index of the current node for the session and the data received from the device in response to the question transmitted by the server to the device. The engine uses the current index value indicating the current node in the interactivity script graph to fetch a text string from a data structure that is the question to transmit to the app. The engine also uses the current index value to retrieve one or more logic rules to apply to the response received from the app. The logic rule will determine the index of the next node in the interactivity script graph for that response from the app. At that point, the current state vector is modified to update the current node location to the determined next node index value. Once the new node location is determined, the traversing engine process loops around to determine the next question to be transmitted to the user.

Top: Get Interactivity Graph ID
Get Patient ID
Determine CurrentNode
Get Question String for CurrentNode
Transmit Question String to User Device
Receive Response from User Device
Get at least one logic rule for CurrentNode
Apply the at least one logic rule on received data to determine NextNode
Set CurrentNode = NextNode
Go to Top
End

Each interactivity script graph can be encoded in a data structure that represents each node as an element in the data structure with sub-elements. In one embodiment, a node has an index label, a text string as a question, at least one logic rule, and for each logic rule, a destination index that is the node in the interactivity script graph the interactivity process moves to if that rule is satisfied. The data structure may include a default next node index if none of the rules are satisfied by the patient’s response. The following is an example data structure for an “Interactivity Graph”, with a “Node” index, one “Rule”, and one “Default” rule:

InteractivityScriptGraph: <Node, QuestionText, Rule <NextNode>, Default<NextNode»

The traversing engine may be implemented using a question function that takes as input the current node value to determine what question to ask:

QuestionString = Question(Interactivity Graph, CurrentNode);

The traversing engine may be implemented as a function that takes as input the current node value and the received text string responding to the question:

NextNode = Traverse(Interactivity Graph, CurrentNode, ReceivedTextString);

The patient state string is then updated:

PatientState.CurrentNode = NextNode;

By interacting with the system the patient completes a series of self-assessments in an interactive graphic-user-interface (GUI) which drives the traverse of an interactivity script graph. In one embodiment, the patient may exhibit several characteristics identified by the series of questions.

The objective is to identify the presence of a clinical factor, which clinical factor is present, and how severe it is. The claimed invention also incorporates data from EHR, and a new data record with the determined characteristics is populated.

In another embodiment, each patient is assigned an “intervention” interactivity script graph based on their “digital phenotype”. Digital phenotype is the characteristics of the interaction between patient and device/internet that correlates with certain mental conditions or behavioral dysfunction. The intervention set is an interactive script. Each response routes or determines the next “suggestion”, “question” or “prompt” that goes out.

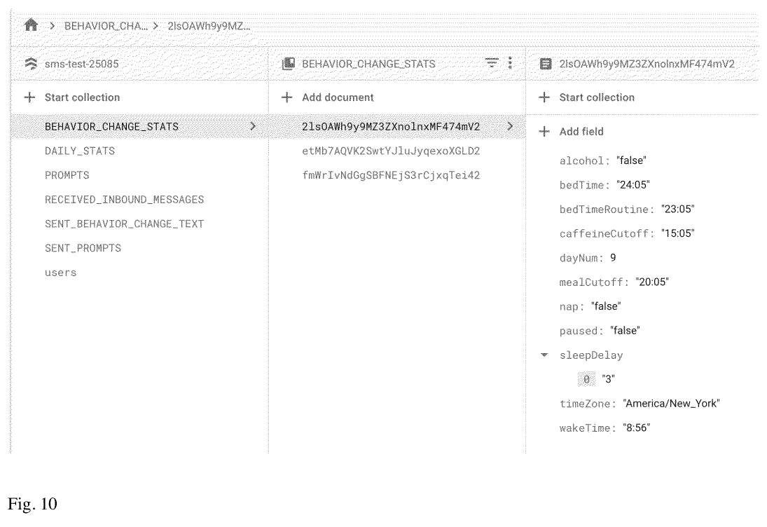

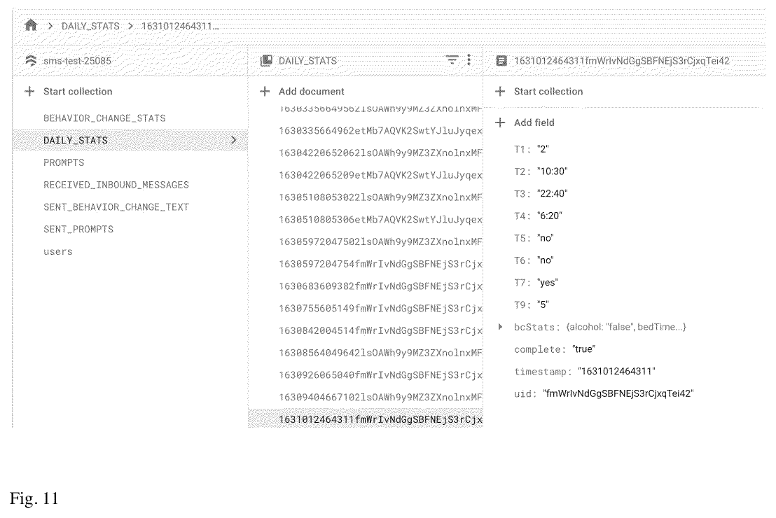

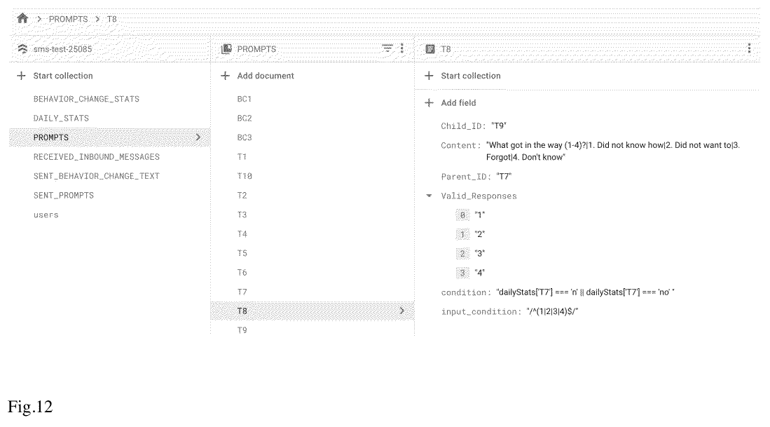

In another embodiment, each node in the interactivity script is encoded as a data record in a database. See FIG. 12. The data record encodes the identity of the node as the filename of the data record. The prompt to be sent the patient is encoded as “content”. The parent node ID is the “parentID” value. A list of valid responses is encoded as well. A condition under which this node would be expressed, i.e. the prompt sent to the user and the response recorded, is encoded in the data record as a set of logical rules. In this example, node T8 would not be expressed unless the patient was already at node T7 and the conditions are evaluated as TRUE. In that case, the content string is transmitted to the user’s devidce, along with the list of valid responses. The client code operating on the server or the user’s device would display on the user’s device the content string as the prompt and the 4 possible valid responses as text or selectable buttons. When a button is actuated or text is sent by the user, the client code sends a message back to the back end server with an identifier corresponding to the user/patient and what the selected option was. This data can be stored in a data record corresponding to the patient, for example the daily statistics for the patient. See FIG. 11. In this data record, each node in the script graph that was activated is listed, along with the corresponding received response. The daily statistic data record may be used to populate a separate database record indicating changes in behavior; See FIG. 10.

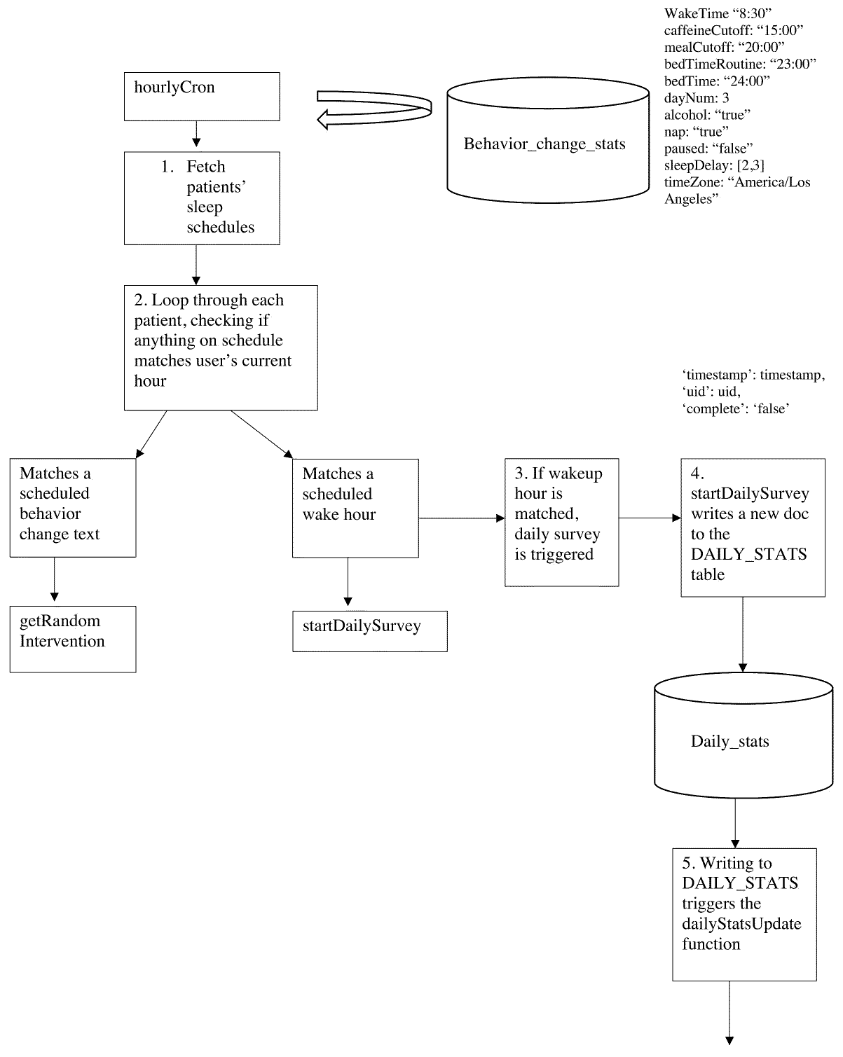

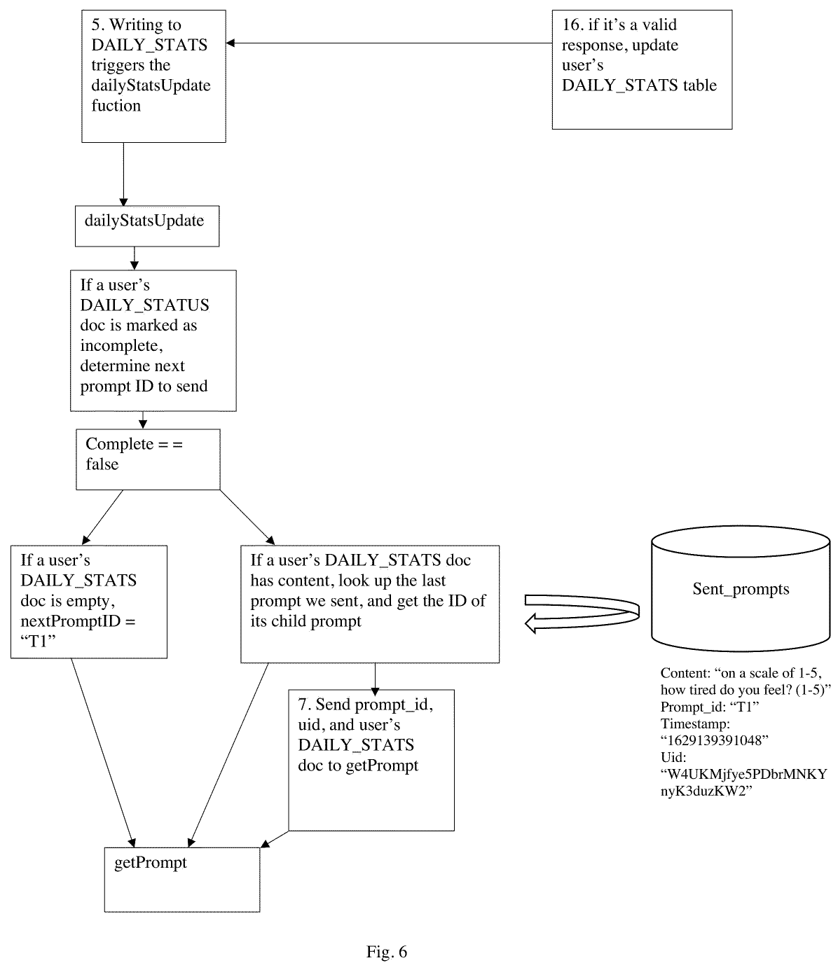

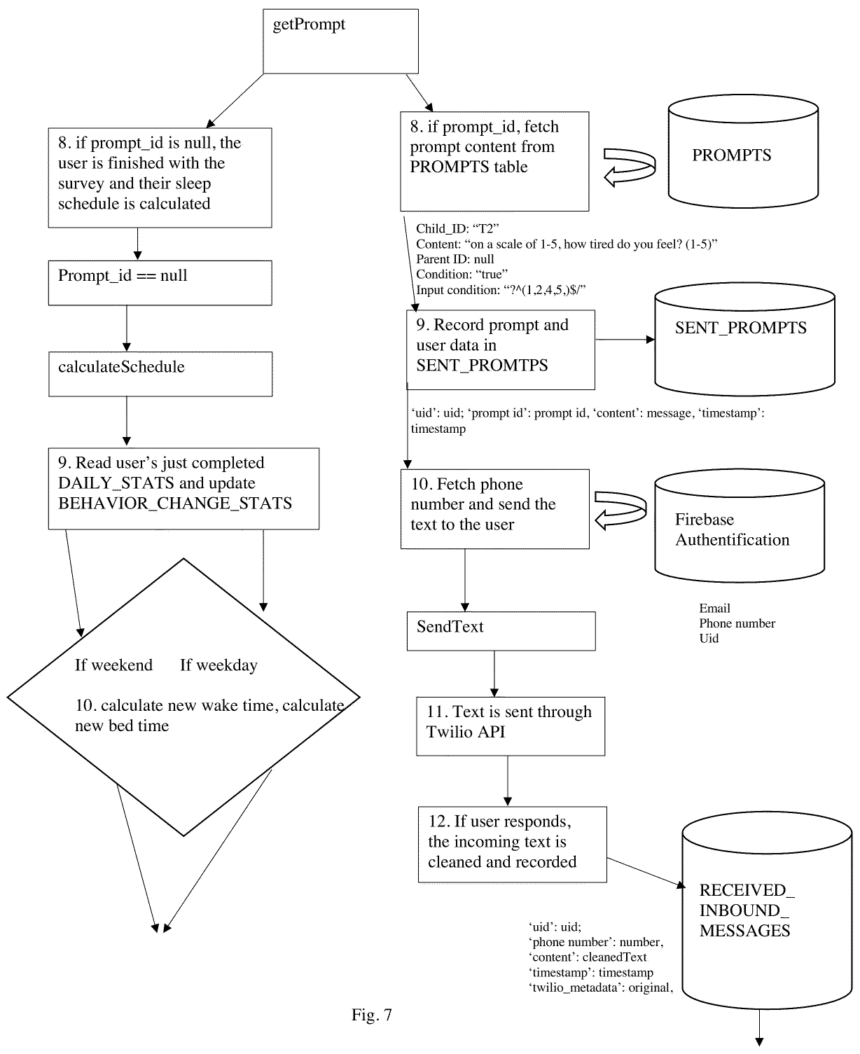

The client-server protocol operates with an application running in the background on the user/patient’s device, or on a server. The app first checks the current time against whether anything in the patient data record shows an activity point in the current hour. FIG. 5. If a wakeup hour is matched within the hour, the system commences a Daily Survey of the patient status. A new daily stats table is created for this session. The interactive script process starts at the root node, in this case labeled as T1. FIG. 6. The label is then passed to the process GetPrompt. The process loops, so if the daily stats table indicates a position further down the graph, the identifier of the next applicable node is determined and data stored from the daily stats table fetched and then passed to GetPrompt. Using the prompt ID, the content of the prompt is selected and transmitted to the patient’s device, along with the range of valid responses. FIG. 7. In addition, a record of the transmission is stored in the Sent Prompts database. The prompts may be sent by text message to the user device, and the text responses received from the user device are stored along with metadata about the received message, including the user identifier, the content, and a time stamp. In this embodiment, the received text messages are cleaned to remove spurious spaces and punctuation and to make the text all small letters.

The received inbound messages are then evaluated. FIG. ,. 9. The scoreinboundmessage processes retrieves the last prompt sent to this user and then gets the prompt ID. The user’s text is then validated against the condition rule called for in the data record corresponding to the promptID. If the received message is determined as one of the valid responses, the response updates the corresponding daily stats table. If a termination point of the interactive script is reached, then the daily stats table is marked as “complete.” Otherwise, the process loops to determine the next node of the interactivity script.

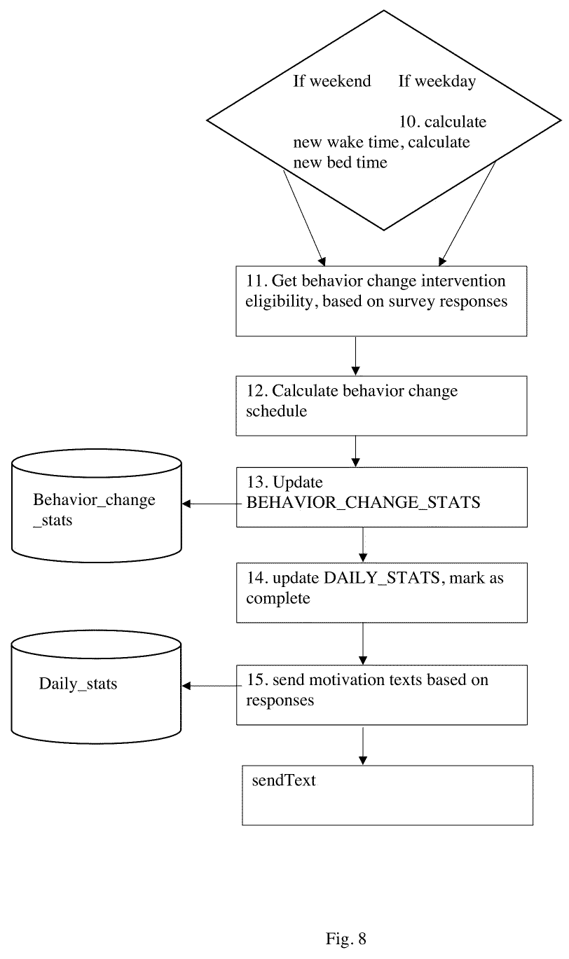

Once the interactivity script reaches a termination point, the behavioral change stats data is updated. FIG. 8. The Daily Stats table for that user/patient is updated to indicate that it is complete. The data from the daily stats table is used to select from a database of possible message content, a content relevant selected in dependence on the daily stats data collected for that patient/user. These may include motivational texts that encourage or discourage certain behaviors. The selected message is then transmitted to the user/patient. These interventions are sent throughout the day at appropriate times according to the user’s schedule, which is calculated based on the responses the user has input that have been received at the server. The user responses are used to calculate times that the intervention messages are transmitted to the user. In this embodiment the system takes as input the text responses from most recent data in the behavioral change data table, and their calculated schedules. The text data is processed to map to one of a set of predetermined category labels. The system then selects an outgoing text message within that mapped category, and transmits it. In one embodiment, the category map is represented by a set of predetermined text messages in a table that for each message has the text string of the message, and a corresponding list of at least one keywords, and the corresponding category label. The incoming messages from the user are scanned for at least one keyword that matches at the relevant time.

In yet another embodiment, severity or presence of a clinical factor determines which script applies. Over time, the interaction will adjust the severity of the clinical risk factor, and move to maintenance. The script moves through the graph, and updates the state of the patient based on the sequence of nodes the process passes through, where each prompt is a node. Certain responses from the user/patient or sequence of responses can be used as input to a correlating function that outputs a score that indicates the relative severity of a situation. This output score can be used to automatically trigger certain intervention activities. One intervention activity is to commence an additional interactivity script. Another activity is to transmit certain motivational messages. A third activity would be to transmit an alert message to another location indicating that a particular patient needs to be contacted by a mental health professional.

In yet another embodiment, the user consents to tracking their broader range of activities on the Internet as a digital phenotype. The detected activity data also is used to determine a route through the script graph. The patient’s current condition is what the patient self-reported, plus where they are in the graph. At a node, transmitted out is a prompt and a response comes back, and all of this data feeds a logic equation that determines which the next node in the graph they go to.

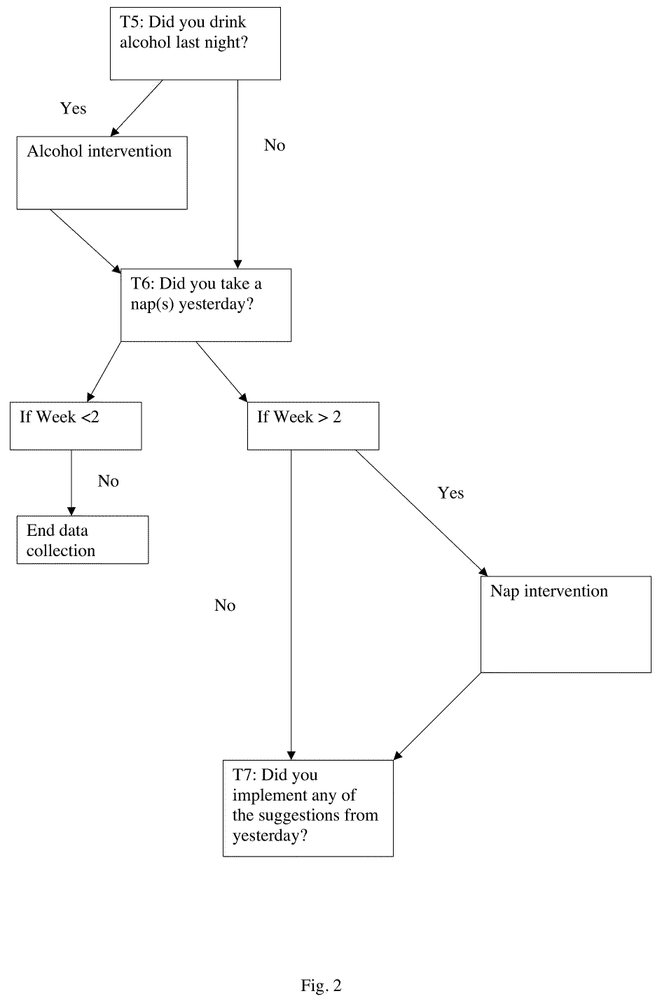

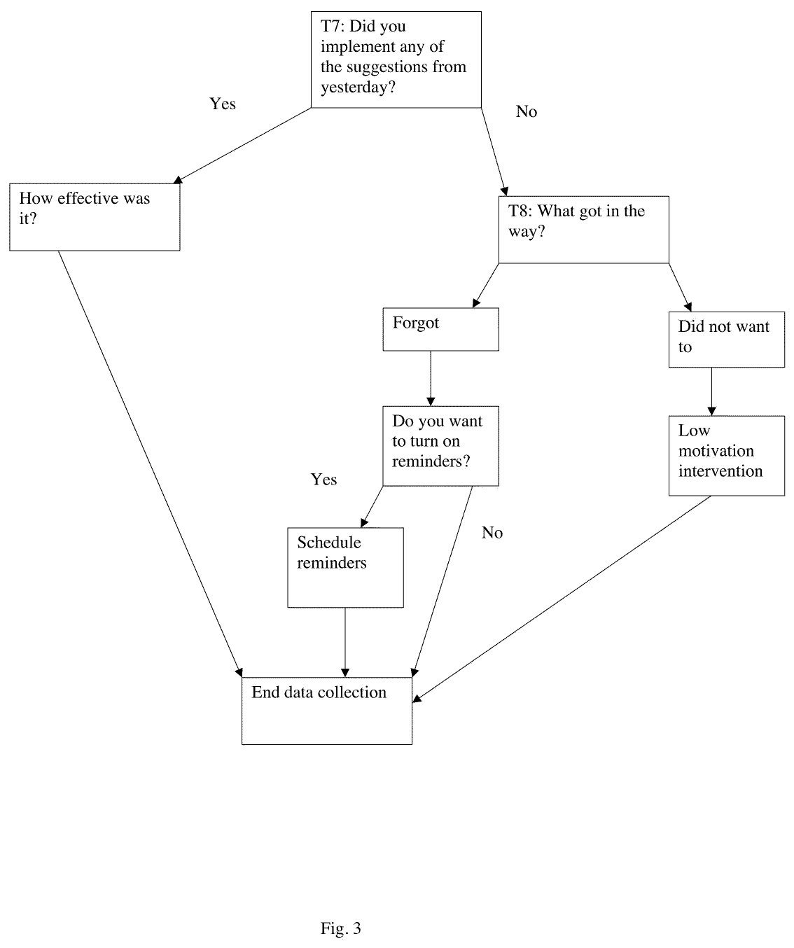

In a further embodiment directed to insomnia tracking, the patient is presented with a series of prompts about their behavioral conditions. For example, if the patient has difficulty sleeping, they are prompted to select a reason why (e.g., worrying/rumination; physical pain, etc.). This then determines which type of intervention should be applied. The patient is further asked a series of questions about alcohol use and napping habits in order to determine which next node in the graph the patient will be directed to. FIG. 2. The patient is then prompted to answer whether any suggestions from the prior day were implemented and if so, how effective they were. If the suggestions were effective, data collection is completed. If not, the patient is presented with further prompts until data collection is complete. FIG. 3.

In another embodiment, the system at the server fetches each user sleep schedule from the users’ data records. The system then loops through the user data to detect the logical condition that there is an entry for a user that matches the current time. If there is a match between a scheduled behavior change text message, the motivational message is transmitted. In one embodiment, the message is selected randomly from a predetermined set of messages that are in a category that matches a category label in the user data record. If the current time matches the wake hour for the user, as indicated in the user data record, then the DailySurvey interactive script is commenced for that day.

### Operating Environment

The system is typically comprised of a central server or servers that are connected by a data network to a user’s computer or device. The central server may be comprised of one or more computers connected to one or more mass storage devices. The precise architecture of the central server does not limit the claimed invention. Further, the user’s computer may be a laptop or desktop type of personal computer. It can also be a cell phone, smart phone or other handheld device, including a tablet. The precise form factor of the user’s computer does not limit the claimed invention. Examples of well-known computing systems, environments, and/or configurations that may be suitable for use with the invention include, but are not limited to, personal computers, server computers, hand-held computers, laptop or mobile computer or communications devices such as cell phones, smart phones, and PDA’s, multiprocessor systems, microprocessor-based systems, set top boxes, programmable consumer electronics, network PCs, minicomputers, mainframe computers, distributed computing environments that include any of the above systems or devices, and the like. Indeed, the terms “computer,” “server,” and the like may be used interchangeably herein, and may refer to any of the above devices and systems.

The user environment may be housed in the central server or operatively connected to it remotely using a network. In one embodiment, the user’s computer is omitted, and instead an equivalent computing functionality is provided that works on a server. In this case, a user would log into the server from another computer over a network and access the system through a user environment, and thereby access the functionality that would in other embodiments, operate on the user’s computer. Further, the user may receive from and transmit data to the central server by means of the Internet, whereby the user accesses an account using an Internet web-browser and browser displays an interactive web page operatively connected to the central server. The server transmits and receives data in response to data and commands transmitted from the browser in response to the customer’s actuation of the browser user interface. Some steps of the invention may be performed on the user’s computer and interim results transmitted to a server. These interim results may be processed at the server and final results passed back to the user computer.

The Internet is a computer network that permits users operating a computer device to interact with computer servers located remotely and to view content that is delivered over the network from the remote servers to the computer device as data files or data streams. In one kind of protocol, the servers present webpages that are rendered on the user’s device using a local program known as a browser. The browser receives one or more data files from the server that are displayed on the user’s computer screen. The browser seeks those data files from a specific address, which is represented by an alphanumeric string called a Universal Resource Locator (URL). However, the webpage may contain components that are downloaded from a variety of URL’s or IP addresses. In addition, the data received from a URL may be a data stream rather than a single file. In one embodiment, the browser interacts with a server using the URL using the HTTP or HTTPS protocol. In addition, the browser may send data to the server denoted by the URL by appending data that gets sent to the server into the HTTP or HTTPS request that is comprised of the URL. The server receiving the HTTP or HTTPS request may use the data payload contained in the request in order to process the request. A website is a collection of related URL’s, typically all sharing the same root address or under the control of some entity. In one embodiment different regions of the simulated space displayed by the browser have different URL’s. That is, the webpage encoding the simulated space can be a unitary data structure, but different URL’s reference different locations in the data structure. The user computer can operate a program that receives from a remote server a data file that is passed to a program that interprets the data in the data file and commands the display device to present particular text, images, video, audio and other objects. In some embodiments, the remote server delivers a data file that is comprised of computer code that the browser program interprets, for example, scripts. The program can detect the relative location of the cursor when the mouse button is actuated, and interpret a command to be executed based on location on the indicated relative location on the display when the button was pressed. The data file may be an HTML document, the program a web-browser program and the command a hyper-link that causes the browser to request a new HTML document from another remote data network address location. The HTML can also have references that result in other code modules being called up and executed, for example, Flash or other native code. Alternatively, the data file returned may conform to a hyper text format like XML.

The invention may also be entirely executed on one or more servers. A server may be a computer comprised of a central processing unit with a mass storage device and a network connection. In addition a server can include multiple of such computers connected together with a data network or other data transfer connection, or, multiple computers on a network with network accessed storage, in a manner that provides such functionality as a group. Practitioners of ordinary skill will recognize that functions that are accomplished on one server may be partitioned and accomplished on multiple servers that are operatively connected by a computer network by means of appropriate inter process communication. In one embodiment, a user’s computer can run an application that causes the user’s computer to transmit a stream of one or more data packets across a data network to a second computer, referred to here as a server. The server, in turn, may be connected to one or more mass data storage devices where the database is stored. In addition, the access of the website can be by means of an Internet browser accessing a secure or public page or by means of a client program running on a local computer that is connected over a computer network to the server. A data message and data upload or download can be delivered over the Internet using typical protocols, including TCP/IP, HTTP, TCP, UDP, SMTP, RPC, FTP or other kinds of data communication protocols that permit processes running on two respective remote computers to exchange information by means of digital network communication. As a result a data message can be one or more data packets transmitted from or received by a computer containing a destination network address, a destination process or application identifier, and data values that can be parsed at the destination computer located at the destination network address by the destination application in order that the relevant data values are extracted and used by the destination application. The precise architecture of the central server does not limit the claimed invention. In addition, the data network may operate with several levels, such that the user’s computer is connected through a fire wall to one server, which routes communications to another server that executes the disclosed methods.

The server can execute a program that receives the transmitted packet and interpret the transmitted data packets in order to extract database query information or to process a request using data comprising the data packets. The server can then execute the remaining steps of the invention by means of accessing the mass storage devices to derive the desired result of the query or requested process. Alternatively, the server can transmit the query information or request processing to another computer that is connected to the mass storage devices, and that computer can execute the invention to derive the desired result. The result can then be transmitted back to the user’s computer by means of another stream of one or more data packets appropriately addressed to the user’s computer. In addition, the user’s computer may obtain data from the server that is considered a website, that is, a collection of data files that when retrieved by the user’s computer and rendered by a program running on the user’s computer, displays on the display screen of the user’s computer text, images, video and in some cases outputs audio. The access of the website can be by means of a client program running on a local computer that is connected over a computer network accessing a secure or public page on the server using an Internet browser or by means of running a dedicated application that interacts with the server, sometimes referred to as an “app.” The data messages may comprise a data file that may be an HTML document (or other hypertext formatted document file, like XML), commands sent between the remote computer and the server and a web-browser program or app running on the remote computer that interacts with the data received from the server. The command can be a hyper-link that causes the browser to request a new HTML document from another remote data network address location. The HTML can also have references that result in other code modules being called up and executed, for example, Flash, scripts or other code. The HTML file may also have code embedded in the file that is executed by the client program as an interpreter, in one embodiment, Javascript. As a result a data message can be a data packet transmitted from or received by a computer containing a destination network address, a destination process or application identifier, and data values or program code that can be parsed at the destination computer located at the destination network address by the destination application in order that the relevant data values or program code are extracted and used by the destination application.

The invention may also be practiced in distributed computing environments where tasks are performed by remote processing devices that are linked through a communications network. In a distributed computing environment, program modules may be located in both local and remote computer storage media including memory storage devices. Practitioners of ordinary skill will recognize that the invention may be executed on one or more computer processors that are linked using a data network, including, for example, the Internet. In another embodiment, different steps of the process can be executed by one or more computers and storage devices geographically separated by connected by a data network in a manner so that they operate together to execute the process steps. In one embodiment, a user’s computer can run an application that causes the user’s computer to transmit a stream of one or more data packets across a data network to a second computer, referred to here as a server. The server, in turn, may be connected to one or more mass data storage devices where the database is stored. The server can execute a program that receives the transmitted packet and interpret the transmitted data packets in order to extract database query information. The server can then execute the remaining steps of the invention by means of accessing the mass storage devices to derive the desired result of the query. Alternatively, the server can transmit the query information to another computer that is connected to the mass storage devices, and that computer can execute the invention to derive the desired result. The result can then be transmitted back to the user’s computer by means of another stream of one or more data packets appropriately addressed to the user’s computer. In one embodiment, a relational database may be housed in one or more operatively connected servers operatively connected to computer memory, for example, disk drives. In yet another embodiment, the initialization of the relational database may be prepared on the set of servers and the interaction with the user’s computer occurs at a different place in the overall process.

The method described herein can be executed on a computer system, generally comprised of a central processing unit (CPU) that is operatively connected to a memory device, data input and output circuitry (IO) and computer data network communication circuitry. Computer code executed by the CPU can take data received by the data communication circuitry and store it in the memory device. In addition, the CPU can take data from the I/O circuitry and store it in the memory device. Further, the CPU can take data from a memory device and output it through the IO circuitry or the data communication circuitry. The data stored in memory may be further recalled from the memory device, further processed or modified by the CPU in the manner described herein and restored in the same memory device or a different memory device operatively connected to the CPU including by means of the data network circuitry. In some embodiments, data stored in memory may be stored in the memory device, or an external mass data storage device like a disk drive. In yet other embodiments, the CPU may be running an operating system where storing a data set in memory is performed virtually, such that the data resides partially in a memory device and partially on the mass storage device. The CPU may perform logic comparisons of one or more of the data items stored in memory or in the cache memory of the CPU, or perform arithmetic operations on the data in order to make selections or determinations using such logical tests or arithmetic operations. The process flow may be altered as a result of such logical tests or arithmetic operations so as to select or determine the next step of a process. For example, the CPU may obtain two data values from memory and the logic in the CPU determines whether they are the same or not. Based on such Boolean logic result, the CPU then selects a first or a second location in memory as the location of the next step in the program execution. This type of program control flow may be used to program the CPU to determine data, or select a data from a set of data. The memory device can be any kind of data storage circuit or magnetic storage or optical device, including a hard disk, optical disk or solid state memory. The IO devices can include a display screen, loudspeakers, microphone and a movable mouse that indicate to the computer the relative location of a cursor position on the display and one or more buttons that can be actuated to indicate a command. The IO device may also be sensors that detect haptic motion, fingers scrolling or touching a screen, fingerprint scanners, cameras or other interactivity mechanisms.

The computer can display on the display screen operatively connected to the I/O circuitry the appearance of a user interface. Various shapes, text and other graphical forms are displayed on the screen as a result of the computer generating data that causes the pixels comprising the display screen to take on various colors and shades or brightness. The user interface may also display a graphical object referred to in the art as a cursor. The object’s location on the display indicates to the user a selection of another object on the screen. The cursor may be moved by the user by means of another device connected by I/O circuitry to the computer. This device detects certain physical motions of the user, for example, the position of the hand on a flat surface or the position of a finger on a flat surface. Such devices may be referred to in the art as a mouse or a track pad. In some embodiments, the display screen itself can act as a trackpad by sensing the presence and position of one or more fingers on the surface of the display screen. When the cursor is located over a graphical object that appears to be a button or switch, the user can actuate the button or switch by engaging a physical switch on the mouse or trackpad or computer device or tapping the trackpad or touch sensitive display. When the computer detects that the physical switch has been engaged (or that the tapping of the track pad or touch sensitive screen has occurred), it takes the apparent location of the cursor (or in the case of a touch sensitive screen, the detected position of the finger) on the screen and executes the process associated with that location. As an example, not intended to limit the breadth of the disclosed invention, a graphical object that appears to be a two dimensional box with the word “enter” within it may be displayed on the screen. If the computer detects that the switch has been engaged while the cursor location (or finger location for a touch sensitive screen) was within the boundaries of a graphical object, for example, the displayed box, the computer will execute the process associated with the “enter” command. In this way, graphical objects on the screen create a user interface that permits the user to control the processes operating on the computer.

In some instances, especially where the user computer is a mobile computing device used to access data through the network the network may be any type of cellular, IP-based or converged telecommunications network, including but not limited to Global System for Mobile Communications (GSM), Time Division Multiple Access (TDMA), Code Division Multiple Access (CDMA), Orthogonal Frequency Division Multiple Access (OFDM), General Packet Radio Service (GPRS), Enhanced Data GSM Environment (EDGE), Advanced Mobile Phone System (AMPS), Worldwide Interoperability for Microwave Access (WiMAX), Universal Mobile Telecommunications System (UMTS), Evolution-Data Optimized (EVDO), Long Term Evolution (LTE), Ultra Mobile Broadband (UMB), Voice over Internet Protocol (VoIP), Unlicensed Mobile Access (UMA), any form of 802.11.xx or Bluetooth.

Computer program logic implementing all or part of the functionality previously described herein may be embodied in various forms, including, but in no way limited to, a source code form, a computer executable form, scripts and various intermediate forms (e.g., forms generated by an assembler, compiler, linker, or locator.) Source code may include a series of computer program instructions implemented in any of various programming languages (e.g., an object code, an assembly language, or a high-level language such as Javascript, C, C++, JAVA, or HTML or scripting languages that are executed by Internet web-browsers) for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

The invention may be described in the general context of computer-executable instructions, such as program modules, being executed by a computer. Generally, program modules include routines, programs, objects, binary components that, when executed by the CPU, perform particular tasks or implement particular abstract data types and when running, may generate in computer memory or store on disk, various data structures. A data structure may be represented in the disclosure as a manner of organizing data, but is implemented by storing data values in computer memory in an organized way. Data structures may be comprised of nodes, each of which may be comprised of one or more elements, encoded into computer memory locations into which is stored one or more corresponding data values that are related to an item being represented by the node in the data structure. The collection of nodes may be organized in various ways, including by having one node in the data structure being comprised of a memory location wherein is stored the memory address value or other reference, or pointer, to another node in the same data structure. By means of the pointers, the relationship by and among the nodes in the data structure may be organized in a variety of topologies or forms, including, without limitation, lists, linked lists, trees and more generally, graphs. The relationship between nodes may be denoted in the specification by a line or arrow from a designated item or node to another designated item or node. A data structure may be stored on a mass storage device in the form of data records comprising a database, or as a flat, parsable file. The processes may load the flat file, parse it, and as a result of parsing the file, construct the respective data structure in memory. In other embodiment, the data structure is one or more relational tables stored on the mass storage device and organized as a relational database.

The computer program and data may be fixed in any form (e.g., source code form, computer executable form, or an intermediate form) either permanently or transitorily in a tangible storage medium, such as a semiconductor memory device (e.g., a RAM, ROM, PROM, EEPROM, or Flash-Programmable RAM), a magnetic memory device (e.g., a diskette or fixed hard disk), an optical memory device (e.g., a CD-ROM or DVD), a PC card (e.g., PCMCIA card, SD Card), or other memory device, for example a USB key. The computer program and data may be fixed in any form in a signal that is transmittable to a computer using any of various communication technologies, including, but in no way limited to, analog technologies, digital technologies, optical technologies, wireless technologies, networking technologies, and internetworking technologies. The computer program and data may be distributed in any form as a removable storage medium with accompanying printed or electronic documentation (e.g., a disk in the form of shrink wrapped software product or a magnetic tape), preloaded with a computer system (e.g., on system ROM or fixed disk), or distributed from a server, website or electronic bulletin board or other communication system (e.g., the Internet or World Wide Web.) It is appreciated that any of the software components of the present invention may, if desired, be implemented in ROM (read-only memory) form. The software components may, generally, be implemented in hardware, if desired, using conventional techniques.

It should be noted that the flow diagrams are used herein to demonstrate various aspects of the invention, and should not be construed to limit the present invention to any particular logic flow or logic implementation. The described logic may be partitioned into different logic blocks (e.g., programs, modules, functions, or subroutines) without changing the overall results or otherwise departing from the true scope of the invention. Oftentimes, logic elements may be added, modified, omitted, performed in a different order, or implemented using different logic constructs (e.g., logic gates, looping primitives, conditional logic, and other logic constructs) without changing the overall results or otherwise departing from the true scope of the invention. Where the disclosure refers to matching or comparisons of numbers, values, or their calculation, these may be implemented by program logic by storing the data values in computer memory and the program logic fetching the stored data values in order to process them in the CPU in accordance with the specified logical process so as to execute the matching, comparison or calculation and storing the result back into computer memory or otherwise branching into another part of the program logic in dependence on such logical process result. The locations of the stored data or values may be organized in the form of a data structure.

The described embodiments of the invention are intended to be exemplary and numerous variations and modifications will be apparent to those skilled in the art. All such variations and modifications are intended to be within the scope of the present invention as defined in the appended claims. Although the present invention has been described and illustrated in detail, it is to be clearly understood that the same is by way of illustration and example only, and is not to be taken by way of limitation. It is appreciated that various features of the invention which are, for clarity, described in the context of separate embodiments may also be provided in combination in a single embodiment. Conversely, various features of the invention which are, for brevity, described in the context of a single embodiment may also be provided separately or in any suitable combination. It is appreciated that the particular embodiment described in the Appendices is intended only to provide an extremely detailed disclosure of the present invention and is not intended to be limiting.

The foregoing description discloses only exemplary embodiments of the invention. Modifications of the above disclosed apparatus and methods which fall within the scope of the invention will be readily apparent to those of ordinary skill in the art. Accordingly, while the present invention has been disclosed in connection with exemplary embodiments thereof, it should be understood that other embodiments may fall within the spirit and scope of the invention as defined by the following claims.

] The system is typically comprised of a central server or servers that are connected by a data network to a user’s computer or device. The central server may be comprised of one or more computers connected to one or more mass storage devices. The precise architecture of the central server does not limit the claimed invention. Further, the user’s computer may be a laptop or desktop type of personal computer. It can also be a cell phone, smart phone or other handheld device, including a tablet. The precise form factor of the user’s computer does not limit the claimed invention. Examples of well-known computing systems, environments, and/or configurations that may be suitable for use with the invention include, but are not limited to, personal computers, server computers, hand-held computers, laptop or mobile computer or communications devices such as cell phones, smart phones, and PDA’s, multiprocessor systems, microprocessor-based systems, set top boxes, programmable consumer electronics, network PCs, minicomputers, mainframe computers, distributed computing environments that include any of the above systems or devices, and the like. Indeed, the terms “computer,” “server,” and the like may be used interchangeably herein, and may refer to any of the above devices and systems.

The user environment may be housed in the central server or operatively connected to it remotely using a network. In one embodiment, the user’s computer is omitted, and instead an equivalent computing functionality is provided that works on a server. In this case, a user would log into the server from another computer over a network and access the system through a user environment, and thereby access the functionality that would in other embodiments, operate on the user’s computer. Further, the user may receive from and transmit data to the central server by means of the Internet, whereby the user accesses an account using an Internet web-browser and browser displays an interactive web page operatively connected to the central server. The server transmits and receives data in response to data and commands transmitted from the browser in response to the customer’s actuation of the browser user interface. Some steps of the invention may be performed on the user’s computer and interim results transmitted to a server. These interim results may be processed at the server and final results passed back to the user computer.

The Internet is a computer network that permits users operating a computer device to interact with computer servers located remotely and to view content that is delivered over the network from the remote servers to the computer device as data files or data streams. In one kind of protocol, the servers present webpages that are rendered on the user’s device using a local program known as a browser. The browser receives one or more data files from the server that are displayed on the user’s computer screen. The browser seeks those data files from a specific address, which is represented by an alphanumeric string called a Universal Resource Locator (URL). However, the webpage may contain components that are downloaded from a variety of URL’s or IP addresses. In addition, the data received from a URL may be a data stream rather than a single file. In one embodiment, the browser interacts with a server using the URL using the HTTP or HTTPS protocol. In addition, the browser may send data to the server denoted by the URL by appending data that gets sent to the server into the HTTP or HTTPS request that is comprised of the URL. The server receiving the HTTP or HTTPS request may use the data payload contained in the request in order to process the request. A website is a collection of related URL’s, typically all sharing the same root address or under the control of some entity. In one embodiment different regions of the simulated space displayed by the browser have different URL’s. That is, the webpage encoding the simulated space can be a unitary data structure, but different URL’s reference different locations in the data structure. The user computer can operate a program that receives from a remote server a data file that is passed to a program that interprets the data in the data file and commands the display device to present particular text, images, video, audio and other objects. In some embodiments, the remote server delivers a data file that is comprised of computer code that the browser program interprets, for example, scripts. The program can detect the relative location of the cursor when the mouse button is actuated, and interpret a command to be executed based on location on the indicated relative location on the display when the button was pressed. The data file may be an HTML document, the program a web-browser program and the command a hyper-link that causes the browser to request a new HTML document from another remote data network address location. The HTML can also have references that result in other code modules being called up and executed, for example, Flash or other native code. Alternatively, the data file returned may conform to a hyper text format like XML.

The invention may also be entirely executed on one or more servers. A server may be a computer comprised of a central processing unit with a mass storage device and a network connection. In addition a server can include multiple of such computers connected together with a data network or other data transfer connection, or, multiple computers on a network with network accessed storage, in a manner that provides such functionality as a group. Practitioners of ordinary skill will recognize that functions that are accomplished on one server may be partitioned and accomplished on multiple servers that are operatively connected by a computer network by means of appropriate inter process communication. In one embodiment, a user’s computer can run an application that causes the user’s computer to transmit a stream of one or more data packets across a data network to a second computer, referred to here as a server. The server, in turn, may be connected to one or more mass data storage devices where the database is stored. In addition, the access of the website can be by means of an Internet browser accessing a secure or public page or by means of a client program running on a local computer that is connected over a computer network to the server. A data message and data upload or download can be delivered over the Internet using typical protocols, including TCP/IP, HTTP, TCP, UDP, SMTP, RPC, FTP or other kinds of data communication protocols that permit processes running on two respective remote computers to exchange information by means of digital network communication. As a result a data message can be one or more data packets transmitted from or received by a computer containing a destination network address, a destination process or application identifier, and data values that can be parsed at the destination computer located at the destination network address by the destination application in order that the relevant data values are extracted and used by the destination application. The precise architecture of the central server does not limit the claimed invention. In addition, the data network may operate with several levels, such that the user’s computer is connected through a fire wall to one server, which routes communications to another server that executes the disclosed methods.

The server can execute a program that receives the transmitted packet and interpret the transmitted data packets in order to extract database query information or to process a request using data comprising the data packets. The server can then execute the remaining steps of the invention by means of accessing the mass storage devices to derive the desired result of the query or requested process. Alternatively, the server can transmit the query information or request processing to another computer that is connected to the mass storage devices, and that computer can execute the invention to derive the desired result. The result can then be transmitted back to the user’s computer by means of another stream of one or more data packets appropriately addressed to the user’s computer. In addition, the user’s computer may obtain data from the server that is considered a website, that is, a collection of data files that when retrieved by the user’s computer and rendered by a program running on the user’s computer, displays on the display screen of the user’s computer text, images, video and in some cases outputs audio. The access of the website can be by means of a client program running on a local computer that is connected over a computer network accessing a secure or public page on the server using an Internet browser or by means of running a dedicated application that interacts with the server, sometimes referred to as an “app.” The data messages may comprise a data file that may be an HTML document (or other hypertext formatted document file, like XML), commands sent between the remote computer and the server and a web-browser program or app running on the remote computer that interacts with the data received from the server. The command can be a hyper-link that causes the browser to request a new HTML document from another remote data network address location. The HTML can also have references that result in other code modules being called up and executed, for example, Flash, scripts or other code. The HTML file may also have code embedded in the file that is executed by the client program as an interpreter, in one embodiment, Javascript. As a result a data message can be a data packet transmitted from or received by a computer containing a destination network address, a destination process or application identifier, and data values or program code that can be parsed at the destination computer located at the destination network address by the destination application in order that the relevant data values or program code are extracted and used by the destination application.

The invention may also be practiced in distributed computing environments where tasks are performed by remote processing devices that are linked through a communications network. In a distributed computing environment, program modules may be located in both local and remote computer storage media including memory storage devices. Practitioners of ordinary skill will recognize that the invention may be executed on one or more computer processors that are linked using a data network, including, for example, the Internet. In another embodiment, different steps of the process can be executed by one or more computers and storage devices geographically separated by connected by a data network in a manner so that they operate together to execute the process steps. In one embodiment, a user’s computer can run an application that causes the user’s computer to transmit a stream of one or more data packets across a data network to a second computer, referred to here as a server. The server, in turn, may be connected to one or more mass data storage devices where the database is stored. The server can execute a program that receives the transmitted packet and interpret the transmitted data packets in order to extract database query information. The server can then execute the remaining steps of the invention by means of accessing the mass storage devices to derive the desired result of the query. Alternatively, the server can transmit the query information to another computer that is connected to the mass storage devices, and that computer can execute the invention to derive the desired result. The result can then be transmitted back to the user’s computer by means of another stream of one or more data packets appropriately addressed to the user’s computer. In one embodiment, a relational database may be housed in one or more operatively connected servers operatively connected to computer memory, for example, disk drives. In yet another embodiment, the initialization of the relational database may be prepared on the set of servers and the interaction with the user’s computer occur at a different place in the overall process.

The method described herein can be executed on a computer system, generally comprised of a central processing unit (CPU) that is operatively connected to a memory device, data input and output circuitry (IO) and computer data network communication circuitry. Computer code executed by the CPU can take data received by the data communication circuitry and store it in the memory device. In addition, the CPU can take data from the I/O circuitry and store it in the memory device. Further, the CPU can take data from a memory device and output it through the IO circuitry or the data communication circuitry. The data stored in memory may be further recalled from the memory device, further processed or modified by the CPU in the manner described herein and restored in the same memory device or a different memory device operatively connected to the CPU including by means of the data network circuitry. In some embodiments, data stored in memory may be stored in the memory device, or an external mass data storage device like a disk drive. In yet other embodiments, the CPU may be running an operating system where storing a data set in memory is performed virtually, such that the data resides partially in a memory device and partially on the mass storage device. The CPU may perform logic comparisons of one or more of the data items stored in memory or in the cache memory of the CPU, or perform arithmetic operations on the data in order to make selections or determinations using such logical tests or arithmetic operations. The process flow may be altered as a result of such logical tests or arithmetic operations so as to select or determine the next step of a process. For example, the CPU may obtain two data values from memory and the logic in the CPU determine whether they are the same or not. Based on such Boolean logic result, the CPU then selects a first or a second location in memory as the location of the next step in the program execution. This type of program control flow may be used to program the CPU to determine data, or select a data from a set of data. The memory device can be any kind of data storage circuit or magnetic storage or optical device, including a hard disk, optical disk or solid state memory. The IO devices can include a display screen, loudspeakers, microphone and a movable mouse that indicate to the computer the relative location of a cursor position on the display and one or more buttons that can be actuated to indicate a command. The IO device may also be sensors that detect haptic motion, fingers scrolling or touching a screen, fingerprint scanners, cameras or other interactivity mechanisms.

The computer can display on the display screen operatively connected to the I/O circuitry the appearance of a user interface. Various shapes, text and other graphical forms are displayed on the screen as a result of the computer generating data that causes the pixels comprising the display screen to take on various colors and shades or brightness. The user interface may also display a graphical object referred to in the art as a cursor. The object’s location on the display indicates to the user a selection of another object on the screen. The cursor may be moved by the user by means of another device connected by I/O circuitry to the computer. This device detects certain physical motions of the user, for example, the position of the hand on a flat surface or the position of a finger on a flat surface. Such devices may be referred to in the art as a mouse or a track pad. In some embodiments, the display screen itself can act as a trackpad by sensing the presence and position of one or more fingers on the surface of the display screen. When the cursor is located over a graphical object that appears to be a button or switch, the user can actuate the button or switch by engaging a physical switch on the mouse or trackpad or computer device or tapping the trackpad or touch sensitive display. When the computer detects that the physical switch has been engaged (or that the tapping of the track pad or touch sensitive screen has occurred), it takes the apparent location of the cursor (or in the case of a touch sensitive screen, the detected position of the finger) on the screen and executes the process associated with that location. As an example, not intended to limit the breadth of the disclosed invention, a graphical object that appears to be a two dimensional box with the word “enter” within it may be displayed on the screen. If the computer detects that the switch has been engaged while the cursor location (or finger location for a touch sensitive screen) was within the boundaries of a graphical object, for example, the displayed box, the computer will execute the process associated with the “enter” command. In this way, graphical objects on the screen create a user interface that permits the user to control the processes operating on the computer.

In some instances, especially where the user computer is a mobile computing device used to access data through the network the network may be any type of cellular, IP-based or converged telecommunications network, including but not limited to Global System for Mobile Communications (GSM), Time Division Multiple Access (TDMA), Code Division Multiple Access (CDMA), Orthogonal Frequency Division Multiple Access (OFDM), General Packet Radio Service (GPRS), Enhanced Data GSM Environment (EDGE), Advanced Mobile Phone System (AMPS), Worldwide Interoperability for Microwave Access (WiMAX), Universal Mobile Telecommunications System (UMTS), Evolution-Data Optimized (EVDO), Long Term Evolution (LTE), Ultra Mobile Broadband (UMB), Voice over Internet Protocol (VoIP), Unlicensed Mobile Access (UMA), any form of 802.11.xx or Bluetooth.

Computer program logic implementing all or part of the functionality previously described herein may be embodied in various forms, including, but in no way limited to, a source code form, a computer executable form, scripts and various intermediate forms (e.g., forms generated by an assembler, compiler, linker, or locator.) Source code may include a series of computer program instructions implemented in any of various programming languages (e.g., an object code, an assembly language, or a high-level language such as Javascript, C, C++, JAVA, or HTML or scripting languages that are executed by Internet web-browsers) for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

The invention may be described in the general context of computer-executable instructions, such as program modules, being executed by a computer. Generally, program modules include routines, programs, objects, binary components that, when executed by the CPU, perform particular tasks or implement particular abstract data types and when running, may generate in computer memory or store on disk, various data structures. A data structure may be represented in the disclosure as a manner of organizing data, but is implemented by storing data values in computer memory in an organized way. Data structures may be comprised of nodes, each of which may be comprised of one or more elements, encoded into computer memory locations into which is stored one or more corresponding data values that are related to an item being represented by the node in the data structure. The collection of nodes may be organized in various ways, including by having one node in the data structure being comprised of a memory location wherein is stored the memory address value or other reference, or pointer, to another node in the same data structure. By means of the pointers, the relationship by and among the nodes in the data structure may be organized in a variety of topologies or forms, including, without limitation, lists, linked lists, trees and more generally, graphs. The relationship between nodes may be denoted in the specification by a line or arrow from a designated item or node to another designated item or node. A data structure may be stored on a mass storage device in the form of data records comprising a database, or as a flat, parsable file. The processes may load the flat file, parse it, and as a result of parsing the file, construct the respective data structure in memory. In other embodiment, the data structure is one or more relational tables stored on the mass storage device and organized as a relational database.

The computer program and data may be fixed in any form (e.g., source code form, computer executable form, or an intermediate form) either permanently or transitorily in a tangible storage medium, such as a semiconductor memory device (e.g., a RAM, ROM, PROM, EEPROM, or Flash-Programmable RAM), a magnetic memory device (e.g., a diskette or fixed hard disk), an optical memory device (e.g., a CD-ROM or DVD), a PC card (e.g., PCMCIA card, SD Card), or other memory device, for example a USB key. The computer program and data may be fixed in any form in a signal that is transmittable to a computer using any of various communication technologies, including, but in no way limited to, analog technologies, digital technologies, optical technologies, wireless technologies, networking technologies, and internetworking technologies. The computer program and data may be distributed in any form as a removable storage medium with accompanying printed or electronic documentation (e.g., a disk in the form of shrink wrapped software product or a magnetic tape), preloaded with a computer system (e.g., on system ROM or fixed disk), or distributed from a server, website or electronic bulletin board or other communication system (e.g., the Internet or World Wide Web.) It is appreciated that any of the software components of the present invention may, if desired, be implemented in ROM (read-only memory) form. The software components may, generally, be implemented in hardware, if desired, using conventional techniques.

It should be noted that the flow diagrams are used herein to demonstrate various aspects of the invention, and should not be construed to limit the present invention to any particular logic flow or logic implementation. The described logic may be partitioned into different logic blocks (e.g., programs, modules, functions, or subroutines) without changing the overall results or otherwise departing from the true scope of the invention. Oftentimes, logic elements may be added, modified, omitted, performed in a different order, or implemented using different logic constructs (e.g., logic gates, looping primitives, conditional logic, and other logic constructs) without changing the overall results or otherwise departing from the true scope of the invention. Where the disclosure refers to matching or comparisons of numbers, values, or their calculation, these may be implemented by program logic by storing the data values in computer memory and the program logic fetching the stored data values in order to process them in the CPU in accordance with the specified logical process so as to execute the matching, comparison or calculation and storing the result back into computer memory or otherwise branching into another part of the program logic in dependence on such logical process result. The locations of the stored data or values may be organized in the form of a data structure.

The described embodiments of the invention are intended to be exemplary and numerous variations and modifications will be apparent to those skilled in the art. All such variations and modifications are intended to be within the scope of the present invention as defined in the appended claims. Although the present invention has been described and illustrated in detail, it is to be clearly understood that the same is by way of illustration and example only, and is not to be taken by way of limitation. It is appreciated that various features of the invention which are, for clarity, described in the context of separate embodiments may also be provided in combination in a single embodiment. Conversely, various features of the invention which are, for brevity, described in the context of a single embodiment may also be provided separately or in any suitable combination. It is appreciated that the particular embodiment described in the Appendices is intended only to provide an extremely detailed disclosure of the present invention and is not intended to be limiting.

The foregoing description discloses only exemplary embodiments of the invention. Modifications of the above disclosed apparatus and methods which fall within the scope of the invention will be readily apparent to those of ordinary skill in the art. Accordingly, while the present invention has been disclosed in connection with exemplary embodiments thereof, it should be understood that other embodiments may fall within the spirit and scope of the invention as defined by the following claims.

What is claimed:

1. A computer system comprised of at least one remote device and a server, said system comprised of program data that when executed causes the computer system to:
receive from the remote device an identifier data representing the identity of a user of the remote device;
an interactivity graph data structure stored in a mass data storage device comprising computer system;
using the received identifier data to determine a data record corresponding to the user comprised of a current node index data value encoding a current node location in the interactivity graph data structure;
read the current node index data value from the determined data record;
use the read node index data value to determine a first node comprising the interactivity graph data structure, said first node comprised of a data element comprised of a prompt script, a set of at least one valid response values and a set of logic rules for determining a next node in the interactivity graph data structure;
transmit to the user’s device the prompt script corresponding to the determined first node;
display the prompt script on the user’s device;
receive from the user’s device a response value;
determine the logical condition that the received response value is one of the at least one valid response values;
automatically determining a second node in the interactivity graph data structure by evaluating at least one of the next node logic rules with the received response and the current node index value as input;
updating the current node index value corresponding to the user to a node index value corresponding to the determined second node.

2. The system of claim 1 where the program code further causes the system to store the received
user response in a log file corresponding to the user.

3. claim 2 further where the program code further causes the system to detect a time stamp value at approximately the time of receipt of the user response and store the detected time stamp value with a reference to the stored user response in the log file corresponding to the user.

4. The system of claim 1 further comprising a haptic sensor in the user’s remote device and where the program code further causes the system to detect a haptic input into the user’s remote device and transmit to the server a data corresponding to the detected haptic input.

5. The system of claim 4 where the haptic sensor detects motion.

6. The system of claim 4 where the haptic sensor detects an image.

7. The system of claim 4 where the haptic sensor detects the user touching the device.

8. The system of claim 1 where the interactivity graph data structure is stored in the computer system as a plurality of data records, each record comprised of a node index label, a text string prompt, at least one next node logic rule and an at least one destination node index label corresponding to the at least one next node logic rules.

9. The system of claim 8 where the plurality of data records comprising the interactivity graph data structure is further comprised of data representing a default next node index value.

10. The system of claim 1 where the program code further causes the system to:
determine that the user response is not a valid response;
in dependence on the determination that the user response is not valid, reading a default next node data value from the data record corresponding to the current node in the interactivity graph data structure; and
updating the current node index value comprising the user data record to the default node index value.

11. The system of claim 1 where the program code further causes the system to:
use the user response to determine and store at least one behavioural change data value corresponding to the user;
use the stored behavioural change data value to determine one of a set of predetermined category labels;
use the one predetermined category labels to select at least one message content corresponding to the one predetermined category label; and
transmit the determined at least one message content to the remote device.

12. The system of claim 11 where the program code further causes the system to determine one of a set of predetermined category labels by scanning the user response to detect at least one predetermined keyword; and using the detected at least one keywords to match in a data structure a corresponding predetermined category label that corresponds to a data record comprised of the detected at least one keywords.

13. The system of claim 1 where the program code further causes the system to:
receive data from the remote device representing a range of activities on the Internet;
using the received data representing a range of activities to determine a digital phenotype value;
use the digital phenotype value as input into the next node logic rules.

14. The system of claim 1 where the program data further causes the system to
Receive the current time;
Receive at least one user data record;
Determine the logical condition that there is an entry for the user that matches the current time;
In dependence on the determined match, determine if there is a match between a scheduled behavior change text message entry in the user data record and in dependence on determining the scheduled behavior match, transmitting a predetermined text message to the device corresponding to the user;
Receive the wake hour corresponding to the user from the user data record;
Determine if the current time matches the user wake hour;
In dependence on the determination that the current time matches the user wake hour, commence a predetermined interactivity script between the server and the user device.
